(19) United States
(12) Patent Application Publication
(10) Pub. No.: US 2025/0205378 A1
(43) Pub. Date: Jun. 26, 2025

(54) FLUID STERILIZATION DEVICE

(71) Applicant: TOYODA GOSEI CO., LTD., Kiyosu-shi (JP)

(72) Inventor: Satoshi WADA, Kiyosu-shi (JP)

(21) Appl. No.: 18/985,891

(22) Filed: Dec. 18, 2024

(30) Foreign Application Priority Data
Dec. 20, 2023 (JP) 2023-215219

(51) Int. Cl. A61L 2/08; A61L 2/10; A61L 9/20; C02F 1/32; C02F 1/72

(52) U.S. Cl. CPC A61L 2/088; A61L 2/084; A61L 2/10; A61L 9/205; C02F 1/325; C02F 1/725; A61L 2202/11; A61L 2202/121; A61L 2202/122; A61L 2209/12; C02F 2201/3222; C02F 2201/3228; C02F 2303/04; C02F 2305/10

(57) ABSTRACT

A fluid sterilization device includes: a flow path tube configured to form a flow path space through which a fluid flows and in which an inlet and an outlet are formed; and a light source unit disposed in the flow path space and configured to emit ultraviolet light into the flow path space. The light source unit includes an ultraviolet light-emitting element that emits ultraviolet light, an accommodation portion that accommodates the ultraviolet light-emitting element, and a window that seals the accommodation portion and transmits the ultraviolet light from the ultraviolet light-emitting element. A surface of the accommodation portion that comes into contact with the fluid is provided with an ultraviolet light photocatalytic film that is excited by ultraviolet light to exert a photocatalytic effect.

### CROSS-REFERENCE TO RELATED APPLICATIONS

This application is based on and claims priority under 35 USC 119 from Japanese Patent Application No. 2023-215219 filed on Dec. 20, 2023, the entire content of which is incorporated herein by reference.

### TECHNICAL FIELD The present disclosure relates to a fluid sterilization device.

### BACKGROUND ART

A sterilization device that sterilizes and inactivates bacteria and viruses in running water by irradiating the water with ultraviolet light is known. A mercury lamp is widely used as a light source.

Since the mercury lamp uses mercury and thus the mercury lamp is highly toxic, there is a problem that the mercury lamp has a large environmental load. Further, there is also a problem that using a mercury lamp results in increase in size of the sterilization device. Therefore, the mercury lamp has been replaced with an ultraviolet LED.

JP2022-173327A, JP2019-18198A, and JP2020-44301A disclose a fluid sterilization device using an ultraviolet LED. JP2022-173327A and JP2019-18198A disclose a configuration in which a columnar light source unit that protrudes from one end toward the other end of a flow path tube is provided.

### SUMMARY OF INVENTION

In the configuration in which the light source unit comes into contact with the fluid to be sterilized as in JP2022-173327A and JP2019-18198A, contamination adheres to the light source unit.

Aspects of the present disclosure relates to providing a fluid sterilization device in which contamination on a light source unit is prevented.

According to an aspect of the present disclosure, there is provided a fluid sterilization device including:
a flow path tube configured to form a flow path space through which a fluid flows and in which an inlet and an outlet are formed; and
a light source unit disposed in the flow path space and configured to emit ultraviolet light into the flow path space, in which
the light source unit includes
   an ultraviolet light-emitting element that emits ultraviolet light,
   an accommodation portion that accommodates the ultraviolet light-emitting element, and
   a window that seals the accommodation portion and transmits the ultraviolet light from the ultraviolet light-emitting element, and
   a surface of the accommodation portion that comes into contact with the fluid is provided with an ultraviolet light photocatalytic film that is excited by ultraviolet light to exert a photocatalytic effect.

According to an aspect of the present disclosure, there is provided a fluid sterilization device including:
a flow path tube configured to form a flow path space through which a fluid flows and in which an inlet and an outlet are formed; and
a light source unit disposed in the flow path space and configured to emit ultraviolet light and visible light into the flow path space, in which
the light source unit includes
   an ultraviolet light-emitting element that emits ultraviolet light,
   a visible light-emitting element that emits visible light,
   an accommodation portion that accommodates the ultraviolet light-emitting element and the visible light-emitting element, and
   a window that seals the accommodation portion and transmits the ultraviolet light from the ultraviolet light-emitting element and the visible light from the visible light-emitting element, and
   a surface of the accommodation portion that comes into contact with the fluid is provided with a visible light photocatalytic film that is excited by visible light to exert a photocatalytic effect. According to an aspect of the present disclosure, there is provided a fluid sterilization device including:
a flow path tube configured to form a flow path space through which a fluid flows and in which an inlet and an outlet are formed;
a light source unit disposed in the flow path space and configured to emit ultraviolet light into the flow path space; and
a second light source unit configured to emit visible light into the flow path space, in which
the light source unit includes
   an ultraviolet light-emitting element that emits ultraviolet light,
   an accommodation portion that accommodates the ultraviolet light-emitting element, and
   a window that seals the accommodation portion and transmits the ultraviolet light from the ultraviolet light-emitting element, and
the second light source unit includes
   a visible light-emitting element that emits visible light,
   a second accommodation portion that accommodates the visible light-emitting element, and
   a second window that seals the second accommodation portion and transmits the visible light from the visible light-emitting element, and
   a surface of the accommodation portion that comes into contact with the fluid is provided with a visible light photocatalytic film that is excited by visible light to exert a photocatalytic effect, and
   a surface of the second accommodation portion that comes into contact with the fluid is provided with an ultraviolet light photocatalytic film that is excited by ultraviolet light to exert a photocatalytic effect.

According to the aspects of the present disclosure, the photocatalytic film is provided on the surface of the accommodation portion that comes into contact with the fluid, and the photocatalytic film is excited by ultraviolet light or visible light to exert a photocatalytic effect.

Therefore, adhesion of contamination to the accommodation portion may be prevented.

### BRIEF DESCRIPTION OF DRAWINGS

Exemplary embodiment(s) of the present invention will be described in detail based on the following figures, wherein:

FIG. 1 is a perspective view illustrating a configuration of a fluid sterilization device according to Embodiment 1;

FIG. 2 is a cross-sectional view illustrating the configuration of the fluid sterilization device according to Embodiment 1 taken along line II-II in FIG. 1;

FIG. 3 is a cross-sectional view illustrating the configuration of the fluid sterilization device according to Embodiment 1 taken along line III-III in FIG. 1;

FIG. 4A is a cross-sectional view illustrating the configuration of the fluid sterilization device according to Embodiment 1 taken along line IVa-IVa in FIG. 1;

FIG. 4B is a cross-sectional view illustrating the configuration of the fluid sterilization device according to Embodiment 1 taken along line IVb-IVb in FIG. 1;

FIG. 5A is a view schematically illustrating a first end portion 100a side and a flow of a fluid;

FIG. 5B is a view schematically illustrating a second end portion 100b side and a flow of the fluid; and

FIGS. 6A, 6B, and 6C are diagrams schematically illustrating a configuration of a light source unit of a fluid sterilization device according to Modification 1 of Embodiment 1.

### DESCRIPTION OF EMBODIMENTS

A first fluid sterilization device includes a flow path tube that forms a flow path space through which a fluid flows and in which an inlet and an outlet are formed, and a light source unit that is disposed in the flow path space and emits ultraviolet light into the flow path space. The light source unit includes an ultraviolet light-emitting element that emits ultraviolet light, an accommodation portion that accommodates the ultraviolet light-emitting element, and a window that seals the accommodation portion and transmits the ultraviolet light from the ultraviolet light-emitting element. A surface of the accommodation portion that comes into contact with the fluid is provided with an ultraviolet light photocatalytic film that is excited by ultraviolet light and exerts a photocatalytic effect.

A second fluid sterilization device includes a flow path tube that forms a flow path space through which a fluid flows and in which an inlet and an outlet are formed, and a light source unit disposed in the flow path space and emits ultraviolet light and visible light into the flow path space. The light source unit includes an ultraviolet light-emitting element that emits ultraviolet light, a visible light-emitting element that emits visible light, an accommodation portion that accommodates the ultraviolet light-emitting element and the visible light-emitting element, and a window that seals the accommodation portion and transmits the ultraviolet light from the ultraviolet light-emitting element and the visible light from the visible light-emitting element. A surface of the accommodation portion that comes into contact with the fluid is provided with a visible light photocatalytic film that is excited by visible light and exerts a photocatalytic effect.

A third fluid sterilization device includes a flow path tube that forms a flow path space through which a fluid flows and in which an inlet and an outlet are formed; a light source unit disposed in the flow path space and emits ultraviolet light into the flow path space, and a second light source unit that emits visible light into the flow path space. The light source unit includes an ultraviolet light-emitting element that emits ultraviolet light, an accommodation portion that accommodates the ultraviolet light-emitting element, and a window that seals the accommodation portion and transmits the ultraviolet light from the ultraviolet light-emitting element. The second light source unit includes a visible light-emitting element that emits visible light, a second accommodation portion that accommodates the visible light-emitting element, and a second window that seals the second accommodation portion and transmits the visible light from the visible light-emitting element. A surface of the accommodation portion that comes into contact with the fluid is provided with a visible light photocatalytic film that is excited by visible light and exerts a photocatalytic effect. A surface of the second 15 accommodation portion that comes into contact with the fluid is provided with an ultraviolet light photocatalytic film that is excited by ultraviolet light and exerts a photocatalytic effect.

In the first fluid sterilization device or the third fluid sterilization device, the flow path space may be provided with an ultraviolet light reflective film that reflects ultraviolet light and irradiates the ultraviolet light photocatalytic film with the reflected ultraviolet light.

In the second fluid sterilization device or the third fluid sterilization device, the flow path space may be provided with a visible light reflective film that reflects visible light and irradiates the visible light photocatalytic film with the reflected visible light.

In the second fluid sterilization device or the third fluid sterilization device, the flow path space may be provided with a reflective film that reflects ultraviolet light and visible light and irradiates the visible light photocatalytic film with the reflected ultraviolet light and visible light.

A surface of the ultraviolet light reflective film may be provided with a second ultraviolet light photocatalytic film that is excited by ultraviolet light and exerts a photocatalytic effect.

A surface of the visible light reflective film may be provided with a photocatalytic film that is excited by ultraviolet light or visible light and exerts a photocatalytic effect.

A surface of the reflective film is provided with a photocatalytic film that is excited by ultraviolet light or visible light and exerts a photocatalytic effect.

### Embodiment 1

### 1. Overview of Configuration of Fluid Sterilization Device 1

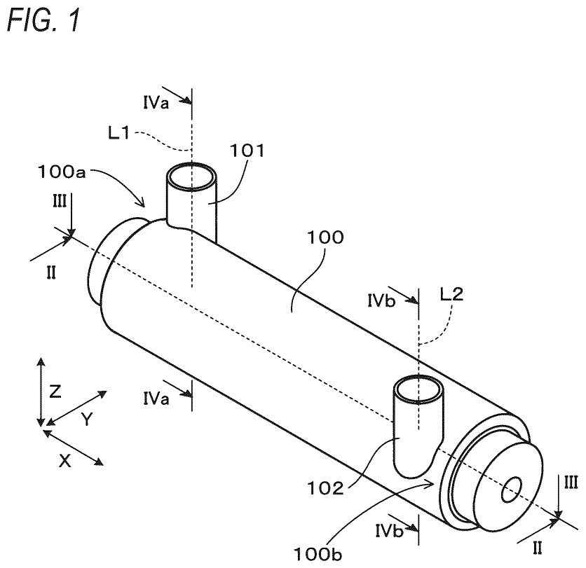

FIG. 1 is a perspective view illustrating a configuration of a fluid sterilization device 1 according to Embodiment 1. As illustrated in FIG. 1, the fluid sterilization device 1 according to Embodiment 1 includes a flow path tube 100 and two light source units 110 therein. In FIG. 1, an X-axis is taken in a direction of a central axis O of the flow path tube 100, a Y-axis is taken in a direction orthogonal to the X-axis and parallel to a central axis L1 of an inlet, and a Z-axis is taken in a direction perpendicular to the X-axis and the Y-axis.

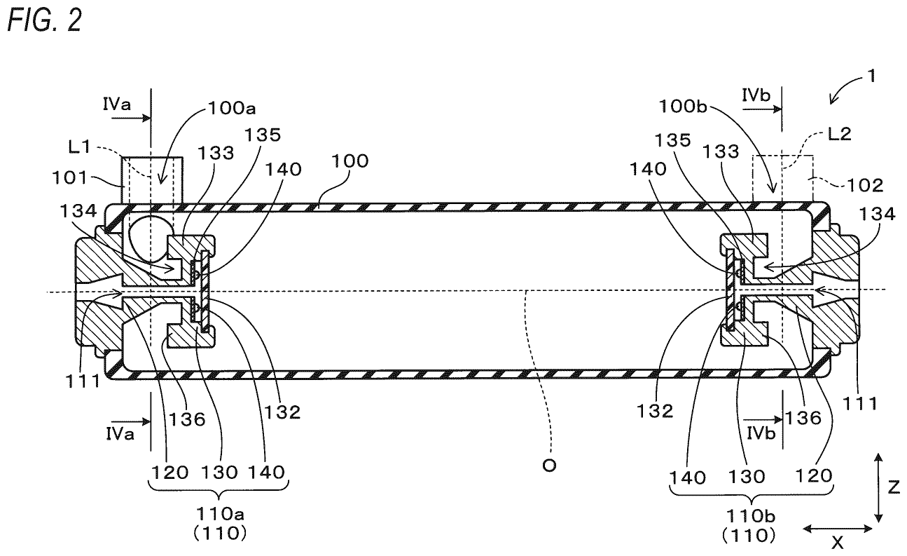

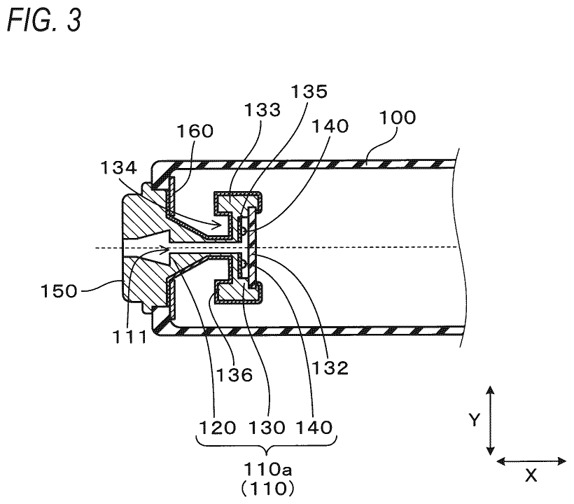

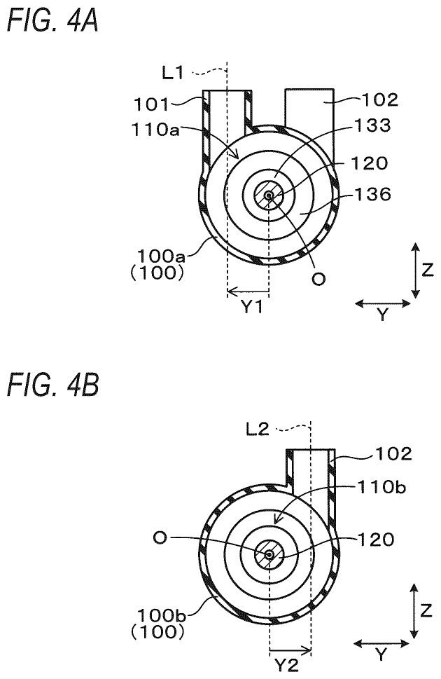

FIG. 2 is a cross-sectional view illustrating the configuration of the fluid sterilization device 1 according to Embodiment 1, and illustrates a cross section (ZX plane) taken along line II-II in FIG. 1. As illustrated in FIG. 2, inside the flow path tube 100, the light source units 110 are respectively disposed at both ends of the flow path tube 100. FIG. 3 is a cross-sectional view illustrating the configuration of the fluid sterilization device according to Embodiment 1, and is a view illustrating a portion of a cross section (XY plane) taken along III-III in FIG. 1 (on a first end portion 100a side). FIGS. 4A and 4B are cross-sectional views illustrating the configuration of the fluid sterilization device 1 according to Embodiment 1. FIG. 4A is a cross-sectional view (YZ plane) taken along IVa-IVa in FIG. 1, and FIG. 4B is a cross-sectional view (YZ plane) taken along IVb-IVb in FIG. 1.

The fluid sterilization device 1 according to Embodiment 1 is a device that causes a fluid to flow from an inlet 101 of the flow path tube 100 to a flow path space inside the flow path tube 100, irradiates the fluid with ultraviolet light from the light source unit 110 to sterilize the fluid, and discharges the sterilized fluid from an outlet 102. The fluid to be sterilized may be a gas or a liquid, and may be a mixture of a gas and a liquid, a mixture of a gas and a powdery solid, or the like as long as the fluid has fluidity. When the fluid is a liquid, examples thereof include water, oil, alcohol, and a solution containing the same as a solvent.

### 2. Details of Each Configuration of Fluid Sterilization Device 1

Next, each configuration of the fluid sterilization device 1 according to Embodiment 1 will be described in detail.

### 2-1. Configuration of Flow Path Tube 100

The flow path tube 100 has a cylindrical tube shape and has a cylindrical column-shaped space therein. This space is a flow path space through which the fluid to be sterilized flows. Both ends of the flow path tube 100 are each provided with a light source unit 110. Here, one end portion of the flow path tube 100 is referred to as a first end portion 100a, and the other end portion of the flow path tube 100 is referred to as a second end portion 100b. One of the two light source units 110 provided on the first end portion 100a side is referred to as a light source unit 110a, and the other of the two light source units 110 provided on a second end portion 100b side is referred to as a light source unit 110b. Further, a side wall of the flow path tube 100 on the first end portion 100a side is provided with the inlet 101, and a side wall of the flow path tube 100 on the second end portion 100b side is provided with the outlet 102. The inlet 101 and the outlet 102 have a cylindrical tube shape and have a flow path region through which the fluid flows.

Examples of a material for the flow path tube 100 include a stainless steel (SUS), titanium, and polytetrafluoroethylene (PTFE). An inner wall surface of a resin material resistant to ultraviolet light may be covered with a material having a high reflectance to ultraviolet light. The resin material having resistance to the ultraviolet light is, for example, vinyl chloride. Further, examples of the material having a high reflectance to the ultraviolet light include aluminum and PTFE. Further, an outer wall surface of a material that transmits ultraviolet light may be covered with a material having a high reflectance to ultraviolet light. Examples of the material that transmits the ultraviolet light include sapphire, ultraviolet-transmitting glass, fluororesin, and acrylic resin.

An inner wall surface of the flow path tube 100 preferably has an arithmetic mean roughness Ra of 0.2 nm to 10 μm. A resistance of the inner wall surface decreases, allowing the flow to be easily maintained. The arithmetic mean roughness Ra is more preferably 0.2 nm to 3 μm, and still more preferably 0.2 nm to 1 μm.

As illustrated in FIG. 4A, the inlet 101 is disposed such that an inflow direction of the fluid that flows in from the inlet 101 is offset from the central axis O of the flow path tube 100. That is, a flow path central axis, which is a central axis of the flow path region of the inlet 101 (hereinafter, simply referred to as a central axis of the inlet 101) L1 coincides with a direction which is parallel to a line intersecting the central axis O of the flow path tube 100 and does not intersect the central axis O of the flow path tube 100. When viewed in cross section as illustrated in FIG. 4A, the central axis L1 of the inlet 101 is disposed offset by Y1 in a Y direction so as not to pass through the central axis O of the flow path tube 100. By offsetting a position of the inlet 101 in this manner, a helical flow may be formed in the flow path space in the flow path tube 100, and a tangential direction of the helical flow is a direction of the central axis L1 of the inlet 101. Further, as illustrated in FIG. 2, the central axis L1 of the inlet 101 and the central axis O form an angle of 90 degrees. The angle does not necessarily have to be 90 degrees and is preferably 80 to 100 degrees.

As illustrated in FIG. 4B, an outflow direction of the outlet 102 is also disposed offset from the central axis O of the flow path tube 100. That is, a flow path central axis, which is a central axis of the flow path region of the outlet 102 (hereinafter, simply referred to as a central axis of the outlet 102) L2 coincides with a direction which is parallel to a line intersecting the central axis O of the flow path tube 100 and does not intersect the central axis O of the flow path tube 100. When viewed in cross section as illustrated in FIG. 4B, the central axis L2 of the outlet 102 is disposed offset by Y2 in a Y direction so as not to pass through the central axis O of the flow path tube 100. Accordingly, the helical flow may be maintained even on an outlet 102 side, and the tangential direction of the helical flow is a direction of the central axis L2 of the outlet 102. Y1 and Y2 may be different from each other. However, Y1 and Y2 are preferably values as close as possible, and particularly preferably the same value.

### 2-2. Configuration of Light Source Unit 110

The light source unit 110 includes LED packages 140, a support portion 120, and an accommodation portion 130. Hereinafter, the light source unit 110a provided on the first end portion 100a side will be described, and the light source unit 110b provided on the second end portion 100b side has the same configuration.

As illustrated in FIG. 2, the support portion 120 protrudes from the first end of the flow path tube 100 toward the second end, and has a truncated cone-shaped portion. A central axis of the support portion 120 coincides with the central axis O of the flow path tube 100. An inclination angle of a side surface of the truncated cone (angle to a bottom surface) is, for example, 30 to 70°. One end of the support portion 120 on a side with a larger diameter is connected to the first end of the flow path tube 100, and one end of the support portion 120 on a side with a smaller diameter is connected to the accommodation portion 130.

A shape of the support portion 120 is not limited to a truncated cone shape, and may be any shape that tapers toward the second end of the flow path tube 100. Although the shape may be tapered in a stepwise manner, it is preferable that the shape is continuously tapered. For example, a truncated pyramid shape may be used. However, in order to form a helical flow, a truncated cone shape is preferable. Further, the entire support portion 120 may not be a truncated cone, and one portion may be a truncated cone and the other portion may be a column. For example, as illustrated in FIG. 1, a side of a front end of the support portion 120 connected to the accommodation portion 130 may have a cylindrical column shape, and the other portion may have a truncated cone shape.

The accommodation portion 130 is connected to the front end of the support portion 120. The accommodation portion 130 accommodates the LED packages 140. The accommodation portion 130 includes a glass plate 132, a base portion 133, and a substrate 135.

The base portion 133 has a cylindrical column-shaped box shape whose upper surface is opened, and an outside bottom surface is connected to the front end of the support portion 120. The substrate 135 is disposed on a bottom surface inside the box, and the LED packages 140 are mounted on the substrate 135. The glass plate 132 is provided on an upper surface of the box and seals an inside of the box. The glass plate 132 is made of a material that transmits the ultraviolet light from the LED packages 140, such as quartz or sapphire. A photocatalytic film that transmits the ultraviolet light may be provided on a surface of the glass plate 132 to inhibit propagation of bacteria and prevent organic contamination on the glass plate 132. The glass plate 132 is not limited to a flat plate, and may be lenticular. For example, the glass plate 132 may be a TIR lens, a fly-eye lens, and a Fresnel lens.

As illustrated in FIG. 2, the base portion 133 is formed to extend from the front end of the support portion 120 to an outside of the support portion 120 in a radial direction over an entire periphery of the front end of the support portion 120. Therefore, a back surface of the base portion 133 is in contact with the flow path space except for a region connected to the support portion 120.

The base portion 133 includes a peripheral wall 136 that protrudes toward the first end in an outer peripheral region of the back surface thereof, and includes a recess 134 surrounded by the back surface of the base portion 133 and the peripheral wall 136. In Embodiment 1, the peripheral wall 136 does not need to be provided over the entire peripheral, and may be partially provided. If the peripheral wall 136 is provided over the entire peripheral, air may be accumulated in the recess 134 and the cooling efficiency may be reduced. Further, it is preferable that the peripheral wall 136 is provided outside the LED packages 140 when viewed from a central axis direction of the flow path tube 100. That is, it is preferable that the LED packages 140 are positioned in a region of the recess 134. The base portion 133 may be more efficiently cooled.

In Embodiment 1, the base portion 133 has a cylindrical column-shaped box shape, and any shape may be used as long as it has a box shape. For example, the base portion 133 may have a square prism-shaped box shape (square shape). However, from a viewpoint of generating a helical flow, it is preferable to form a cylindrical column-shaped box as that in Embodiment 1.

A material for the support portion 120 and the base portion 133 is titanium. As illustrated in FIG. 3, a photocatalytic film 150 is provided in regions of a surface of the support portion 120 and a surface of the base portion 133 that come into contact with the fluid. Specifically, the regions that come into contact with the fluid include an upper surface, a side surface, and the back surface of the base portion 133, a side surface of the support portion 120, and the like.

The photocatalytic film 150 is made of titanium oxide. The photocatalytic film 150 is formed by oxidizing the surfaces of the support portion 120 and the base portion 133, which are made of titanium. Since the photocatalytic film 150 of the light source unit 110a on a first end side is irradiated with ultraviolet light from the light source unit 110b on a second end side, the photocatalytic film 150 exerts a photocatalytic effect. As a result, propagation of bacteria and adhesion of contamination in regions of the support portion 120 and the base portion 133 that come into contact with the fluid may be prevented.

The material for the support portion 120 and the base portion 133 is not limited to titanium, and the photocatalytic film 150 is also not limited to a surface oxide film. For example, a metal material having high thermal conductivity such as SUS or aluminum, or a high heat dissipation resin mixed with a thermally conductive filler may be used. The photocatalytic film 150 may be formed by sputtering or a sol-gel method. Titanium oxide may be mixed with fluororesin or the like and applied. A material for the photocatalytic film 150 is not limited to titanium oxide, and iron oxide, zinc oxide, tungsten oxide, or the like may be used.

Further, some of a plurality of installed LED packages 140 may be replaced with those for exciting the photocatalytic film 150. For example, a material that is excited by visible light and exerts a photocatalytic effect (for example, copper) may be used as the material for the photocatalytic film 150, and one of the plurality of LED packages 140 may be equipped with a light-emitting element that emits visible light.

As the light-emitting element that emits visible light, for example, an LED package 140 having an emission wavelength of 385 nm or more may be used. Since the LED package 140 having an emission wavelength of 385 nm or more has higher emission efficiency than that of ultraviolet light emission, the photocatalytic effect of the photocatalytic film 150 made of Cu may be more efficiently exerted. Naturally, in this case, the glass plate 132 is made of a material that transmits both ultraviolet light and visible light. Further, since a material for a reflective film 160 described later may be any material that reflects visible light, a range of material selection is widened.

The reflective film 160 that reflects ultraviolet light from the light source unit 110b on the second end side is provided on an end surface of the flow path tube 100 on the first end side. By providing the reflective film 160, an amount of ultraviolet light irradiated to the photocatalytic film 150 may be increased, and the photocatalytic effect of the photocatalytic film 150 may be more effectively exerted. Further, an amount of ultraviolet light irradiated to the fluid may also be increased, and the sterilization efficiency may be further improved. The material for the reflective film 160 may be any material capable of reflecting ultraviolet light, such as aluminum, magnesium, SUS, PTFE, titanium, or resin such as fluorine-coated vinyl chloride.

When the photocatalytic film 150 is made of a material excited by visible light, a material that reflects visible light may be used for the reflective film 160. In particular, a material that reflects both visible light and ultraviolet light is preferable. In addition to increasing an amount of visible light irradiated to the photocatalytic film 150, an amount of ultraviolet light irradiated to the fluid may be increased. For example, aluminum is suitable because aluminum has a high reflectance for both visible light and ultraviolet light.

A photocatalytic film may be provided on a surface of the reflective film 160. Since the reflective film 160 also comes into contact with the fluid, there is a possibility that contamination adheres to the reflective film 160, but this may be prevented by providing the photocatalytic film. A material and a formation method for the photocatalytic film formed on the reflective film 160 are the same as those of the reflective film 160. When a material excited by ultraviolet light is used as the photocatalytic film 150, a material excited by ultraviolet light is used for the photocatalytic film on the reflective film 160. When a material that is excited by visible light is used as the photocatalytic film 150, the photocatalytic film on the reflective film 160 may be made of a material excited by ultraviolet light or a material excited by visible light.

Although the light source unit 110a and the light source unit 110b have the same configuration in Embodiment 1, the photocatalytic film 150 of the light source unit 110a may be made of a material excited by ultraviolet light, the photocatalytic film 150 of the light source unit 110b may be made of a material excited by visible light, and the light source unit 110a may be equipped with LED packages 140 that emit visible light, whereas the light source unit 110b may not be equipped with LED packages 140 that emit visible light. Alternatively, a reverse configuration may also be used.

The LED packages 140 are mounted on the substrate 135. A plurality of LED packages 140 may be mounted. Two LED packages 140 are mounted in FIG. 2. The LED package 140 includes an LED, a substrate on which the LED is mounted, and a lens that seals the LED.

The LED is a light-emitting element that emits ultraviolet light. A wavelength of the ultraviolet light is preferably 250 to 285 nm, which is a wavelength having high sterilization efficiency. A plurality of LEDs may be provided in one LED package 140.

It is preferable that the LED package 140 is mounted in a region outside the support portion 120 when viewed from the central axis direction of the flow path tube 100. Since the fluid may be brought into contact with a region of the back surface of the base portion 133 directly below the LED package 140, the accommodation portion 130 may be efficiently cooled.

In Embodiment 1, the packaged LED package 140 is mounted on the substrate 135. The LED may be directly mounted on the substrate 135.

A through continuous hole 111 is provided at a center of the support portion 120 and the accommodation portion 130. The hole 111 is a hole through which a wiring cable that supplies power to the LED package 140 and circuit components on a mounting substrate is inserted. The wiring cable is drawn into the mounting substrate through the hole 111.

### 3. Regarding Flow Path of Fluid

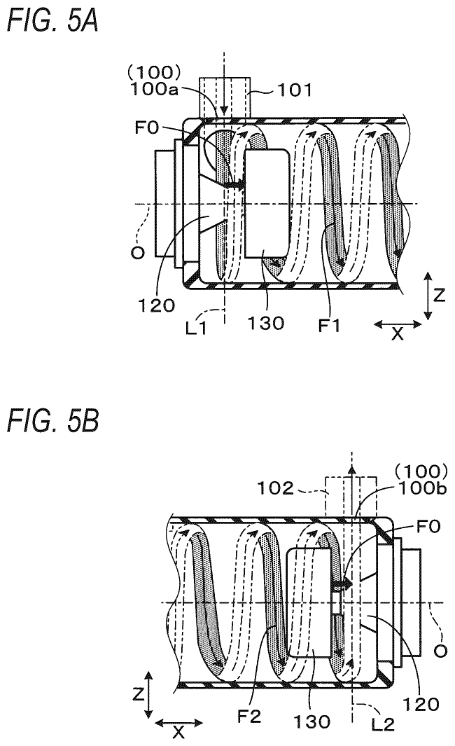

A flow path of the fluid flowing in the flow path tube 100 will be described with reference to FIGS. 5A and 5B. FIG. 5A is a view schematically illustrating a flow path on the first end 100a side (inlet 101 side) of the flow path tube 100, and FIG. 5B is a view schematically illustrating a flow path on the second end 100b side (outlet 102 side) of the flow path tube 100.

As illustrated in FIG. 5A, the fluid that enters the flow path space in the flow path tube 100 from the inlet 101 flows to circulate around the support portion 120 of the light source unit 110a. This is because the inlet 101 is offset from the central axis O of the flow path tube 100, and a position, a shape, and a size of the inlet 101 are set to circulate around the support portion 120. When viewed in the direction of the central axis O of the flow path tube 100, that is, the direction from the first end portion 100a toward the second end portion 100b, the fluid flows to rotate counterclockwise.

The fluid that circulates around the support portion 120 hits the side surface of the truncated conical portion of the support portion 120. Therefore, the fluid is reflected in an axial direction due to an inclination of the side surface, and a flow path toward the accommodation portion 130 is formed. Therefore, the fluid may be efficiently brought into contact with the accommodation portion 130, and the cooling efficiency may be improved.

Further, since the base portion 133 is formed to extend from the front end of the support portion 120 to the outside of the support portion 120 in the radial direction along the entire periphery of the front end of the support portion 120, the fluid may be brought into contact with the back surface of the base portion 133. In particular, the fluid is brought into contact with the region of the back surface of the base portion 133 which is directly below the LED package 140. Therefore, the base portion 133 may be efficiently cooled.

Further, since the peripheral wall 136 is provided on the back surface of the base portion 133 and the recess 134 surrounded by the peripheral wall 136 exists, the fluid easily remains on the back surface of the base portion 133. Therefore, heat may be efficiently conducted from the back surface of the base portion 133 to the fluid, and the cooling efficiency may be improved.

Thereafter, the fluid flows in the central axis direction while rotating around the central axis O in a ring-shaped region between the accommodation portion 130 and the inner wall surface of the flow path tube 100. As a result, a helical flow F1 is formed. By forming the helical flow F1, residence time of the fluid in the flow path space is increased, and irradiation time of the ultraviolet light to the fluid is increased, thereby improving the sterilization efficiency.

On the other hand, on the second end portion 100b side, a helical flow F2 is maintained as illustrated in FIG. 5B. This is because a clean helical flow F1 is formed on the first end portion 100a side, and the helical flow F1 is less likely to collapse even at a distant position. Therefore, the irradiation time of ultraviolet light is longer on the second end portion 100b side as well, and the sterilization efficiency may be improved.

Further, on the second end portion 100b side, the fluid passes through the ring-shaped region between the accommodation portion 130 of the light source unit 110b and the inner wall surface of the flow path tube 100 in the direction of the central axis O while rotating around the central axis O. Then, the fluid flows out from the outlet 102 while circulating around the support portion 120 of the light source unit 110b. Similarly to the inlet 101, the outlet 102 is also offset from the central axis O of the flow path tube 100, and a position, a shape, and a size of the outlet 102 are set to circulate around the support portion 120, so that the fluid can smoothly flow out from the outlet 102, reducing pressure loss.

A portion of the fluid that circulates around the support portion 120 of the light source unit 110b is reflected by the side surface of the support portion 120, forming a flow path FO toward the accommodation portion 130 of the light source unit 110b. Therefore, the accommodation portion 130 of the light source unit 110b may be efficiently cooled.

### 5. Conclusion

As described above, in the fluid sterilization device 1 according to Embodiment 1, the photocatalytic film 150 that is excited by the light from the light source unit 110 and exerts the photocatalytic effect is provided in the regions of the surface of the support portion 120 and the surface of the base portion 133 that come into contact with the fluid. Therefore, adhesion of contamination to the support portion 120 and the base portion 133 may be prevented.

### Modification 1 of Embodiment 1

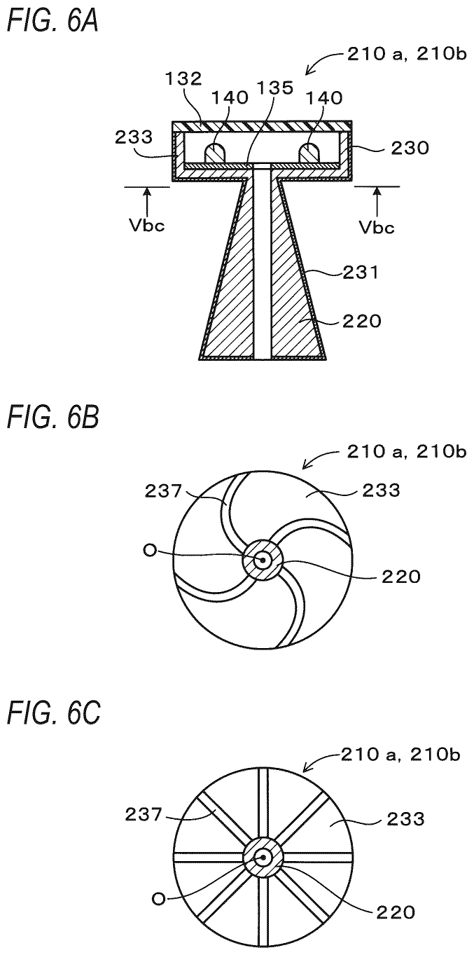

FIGS. 6A, 6B, and 6C are views schematically illustrating a configuration of a light source unit 210 of a fluid sterilization device according to Modification 1 of Embodiment 1. As illustrated in FIG. 6A, the light source unit 210 includes a support portion 220 and an accommodation portion 230. The support portion 220 has the same configuration as the support portion 120 according to Embodiment 1. The accommodation portion 230 has a configuration in which the base portion 133 of the accommodation portion 130 according to Embodiment 1 is replaced with a base portion 233, and other configurations are the same as those of the accommodation portion 130. The base portion 233 has a configuration in which the peripheral wall 136 is eliminated from the base portion 133, and an outer peripheral region on a back surface of the base portion 233 is flat.

A photocatalytic film 231 is provided on surfaces of the base portion 233 and the support portion 220 that come into contact with the fluid. The photocatalytic film 231 is made of the same material as the photocatalytic film 150 in Embodiment 1. The photocatalytic film 231 may prevent adhesion of contamination to the base portion 233 and the support portion 220.

Further, in Modification 1 of Embodiment 1, although the effect of retaining the fluid on the back surface of the accommodation portion 230 is not obtained by the peripheral wall 136, other effects may be obtained in the same manner as in Embodiment 1.

In Modification 1 of Embodiment 1, in the light source unit 210 on the inlet 101 side, a groove 237 may be provided on the back surface of the accommodation portion 230 (back surface of the base portion 233), and the fluid may be guided from a center side to an outer periphery side of the back surface of the accommodation portion. Alternatively, a wall-shaped protrusion may be provided instead of the groove 237.

FIGS. 6B and 6C are each a sectional view illustrating a cross section taken along line Vbc-Vbc in FIG. 6A. FIG. 6B illustrates a case in which a helical groove 237 is provided on the back surface of the base portion 233. A center of the helical is a center of the support portion 220. By providing such helical groove 237, contact time between the fluid and the accommodation portion 230 is increased, so that the accommodation portion 230 may be efficiently cooled. Further, a flow path swirling toward the outer periphery of the back surface of the accommodation portion 230 may be formed, and the fluid passing between an inner wall of the flow path tube 100 and the accommodation portion 230 easily forms a helical flow.

FIG. 6C illustrates a case in which a radial groove 237 is provided on the back surface of the base portion 233. By providing such groove 237, the fluid may be guided to the outer periphery.

The light source unit 210 on the outlet 102 side may also be provided with the groove 237 as illustrated in FIGS. 6 B and 6C. The accommodation portion 230 may be efficiently cooled. Further, the fluid that passes between the inner wall of the flow path tube 100 and the accommodation portion 230 may be guided to the support portion 220, and then a smooth flow path toward the outlet 102 may be formed through reflection by the support portion 220.

### Modification 2 of Embodiment 1

The support portion 120 may have a cylindrical column shape. Although there is no effect of causing the fluid to an accommodation portion 130, other effects may be obtained in the same manner as in Embodiment 1. The accommodation portion 130 may be the same as that in Embodiment 1.

### Modification 3 of Embodiment 1

The back surface of the base portion 133 may coincide with the front end of the support portion 120, and no portion may protrude outside the support portion 120 in the radial direction. Although the fluid cannot be brought into contact with the back surface of the accommodation portion 130 for cooling, other effects may be obtained in the same manner as in Embodiment 1.

### Modification 4 of Embodiment 1

A light intensity sensor may be provided at a center portion of the flow path tube 100. The light intensity sensor is a sensor that detects an intensity of the ultraviolet light in the central portion of the flow path tube 100. For example, outputs of the two light source units 110 are controlled such that the intensity of the ultraviolet light in the central portion is equal to or greater than a predetermined value or more.

Further, the light intensity sensor also serves as a flow guide plate. The light intensity sensor is a wall-shaped protrusion that is provided on the inner wall of the flow path tube 100 and protrudes toward the central axis of the flow path tube 100. The light intensity sensor has a wall shape along a direction of the helical flow, thereby allowing the helical flow to be maintained in the central portion of the flow path tube 100.

### Modification 5 of Embodiment 1

A helical groove 700 may be provided in the inner wall of the flow path tube 100. By providing the helical groove 700 in the flow path tube 100, the helical flow may be easily maintained in the flow path space, and the sterilization efficiency may be improved.

### Other Modifications

In the fluid sterilization device 1 according to Embodiment 1, the light source units 110a, 110b are respectively provided on the inlet 101 side and the outlet 102 side. The light source unit 110a may be provided only on the inlet 101 side when the flow path tube 100 is short. In this case, the reflective film 160 that reflects light from the light source unit 110a may be disposed on an end surface of the flow path tube 100 on the second end side. The light from the light source unit 110a may be reflected by the reflective film 160, and the sterilization efficiency may be improved by irradiating the fluid with the reflected light. Further, the photocatalytic film 150 of the light source unit 110a may be irradiated with the reflected light, and the photocatalytic effect may be further enhanced.

The light source unit 110b may be provided only on the outlet 102 side. In this case as well, the sterilization efficiency may be improved by providing the reflective film 160 on the end surface of the flow path tube 100 on the first end side. Further, the photocatalytic effect of the photocatalytic film 150 of the light source unit 110b may be further enhanced.

In Embodiment 1, the flow path tube 100 has a straight tube shape. However, the flow path tube 100 may have other shapes, such as a U-shaped tube or other tubular shapes, or even a spherical shape. The positions of the inlet 101 and the outlet 102 may be any position. The light source unit 110 may also be at any position within the flow path space.

What is claimed is:

1. A fluid sterilization device comprising:
a flow path tube configured to form a flow path space through which a fluid flows and in which an inlet and an outlet are formed; and
a light source unit disposed in the flow path space and configured to emit ultraviolet light into the flow path space, wherein
the light source unit includes
an ultraviolet light-emitting element that emits ultraviolet light,
an accommodation portion that accommodates the ultraviolet light-emitting element, and
a window that seals the accommodation portion and transmits the ultraviolet light from the ultraviolet light-emitting element, and
a surface of the accommodation portion that comes into contact with the fluid is provided with an ultraviolet light photocatalytic film that is excited by ultraviolet light to exert a photocatalytic effect.

2. A fluid sterilization device comprising:
a flow path tube configured to form a flow path space through which a fluid flows and in which an inlet and an outlet are formed; and
a light source unit disposed in the flow path space and configured to emit ultraviolet light and visible light into the flow path space, wherein
the light source unit includes
an ultraviolet light-emitting element that emits ultraviolet light,
a visible light-emitting element that emits visible light,
an accommodation portion that accommodates the ultraviolet light-emitting element and the visible light-emitting element, and
a window that seals the accommodation portion and transmits the ultraviolet light from the ultraviolet light-emitting element and the visible light from the visible light-emitting element, and
a surface of the accommodation portion that comes into contact with the fluid is provided with a visible light photocatalytic film that is excited by visible light to exert a photocatalytic effect.

3. A fluid sterilization device comprising:
a flow path tube configured to form a flow path space through which a fluid flows and in which an inlet and an outlet are formed;
a light source unit disposed in the flow path space and configured to emit ultraviolet light into the flow path space; and
a second light source unit configured to emit visible light into the flow path space, wherein
the light source unit includes
an ultraviolet light-emitting element that emits ultraviolet light,
an accommodation portion that accommodates the ultraviolet light-emitting element, and
a window that seals the accommodation portion and transmits the ultraviolet light from the ultraviolet light-emitting element, and the second light source unit includes
a visible light-emitting element that emits visible light,
a second accommodation portion that accommodates the visible light-emitting element, and
a second window that seals the second accommodation portion and transmits the visible light from the visible light-emitting element, and
a surface of the accommodation portion that comes into contact with the fluid is provided with a visible light photocatalytic film that is excited by visible light to exert a photocatalytic effect, and
a surface of the second accommodation portion that comes into contact with the fluid is provided with an ultraviolet light photocatalytic film that is excited by ultraviolet light to exert a photocatalytic effect.

4. The fluid sterilization device according to claim 1, wherein
the flow path space is provided with an ultraviolet light reflective film that reflects ultraviolet light and irradiates the ultraviolet light photocatalytic film with the reflected ultraviolet light.

5. The fluid sterilization device according to claim 2, wherein
the flow path space is provided with a visible light reflective film that reflects visible light and irradiates the visible light photocatalytic film with the reflected visible light.

6. The fluid sterilization device according to claim 2, wherein
the flow path space is provided with a reflective film that reflects ultraviolet light and visible light and irradiates the visible light photocatalytic film with the reflected ultraviolet light and visible light.

7. The fluid sterilization device according to claim 4, wherein
a surface of the ultraviolet light reflective film is provided with a second ultraviolet light photocatalytic film that is excited by ultraviolet light to exert a photocatalytic effect.

8. The fluid sterilization device according to claim 5, wherein
a surface of the visible light reflective film is provided with a photocatalytic film that is excited by ultraviolet light or visible light to exert a photocatalytic effect.

9. The fluid sterilization device according to claim 6, wherein
a surface of the reflective film is provided with a photocatalytic film that is excited by ultraviolet light or visible light to exert a photocatalytic effect.

10. The fluid sterilization device according to claim 3, wherein
the flow path space is provided with an ultraviolet light reflective film that reflects ultraviolet light and irradiates the ultraviolet light photocatalytic film with the reflected ultraviolet light.

11. The fluid sterilization device according to claim 3, wherein
the flow path space is provided with a visible light reflective film that reflects visible light and irradiates the visible light photocatalytic film with the reflected visible light.

12. The fluid sterilization device according to claim 3, wherein
the flow path space is provided with a reflective film that reflects ultraviolet light and visible light and irradiates the visible light photocatalytic film with the reflected ultraviolet light and visible light.

13. The fluid sterilization device according to claim 10, wherein
a surface of the ultraviolet light reflective film is provided with a second ultraviolet light photocatalytic film that is excited by ultraviolet light to exert a photocatalytic effect.

14. The fluid sterilization device according to claim 11, wherein
a surface of the visible light reflective film is provided with a photocatalytic film that is excited by ultraviolet light or visible light to exert a photocatalytic effect.

15. The fluid sterilization device according to claim 12, wherein
a surface of the reflective film is provided with a photocatalytic film that is excited by ultraviolet light or visible light to exert a photocatalytic effect.
